(19) United States
(12) Patent Application Publication
(10) Pub. No.: US 2007/0142255 A1
(43) Pub. Date: Jun. 21, 2007

(54) Skin benefit composition and a method for using the same

(75) Inventor: Qiang Qiu, Trumbull, CT (US)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(73) Assignee: Conopco, Inc., d/b/a Unilever

(21) Appl. No.: 11/303,482

(22) Filed: Dec. 16, 2005

(51) Int. Cl. A61K 8/00

(52) U.S. Cl. 510/130; 510/156

(57) ABSTRACT

The present invention is directed to a skin benefit composition having a mixture of fatty acids where at least about 40% of the mixture of fatty acids is C₁₇ or less and from about 8.5 to about 60% is C₁₈-C₂₀. The skin benefit composition of the present invention minimizes the effects of sebum, and moisturizes and lightens skin.

### FIELD OF THE INVENTION

The present invention is directed to a skin benefit composition and a method for improving skin characteristics by using the same. More particularly, the invention is directed to a skin benefit composition comprising a mixture of fatty acids whereby at least about 40% of the total weight of fatty acids used in the composition are C₁₇ or less. The skin benefit composition of the present invention, at the very least and surprisingly, decreases the impact of sebum secretion from sebocytes, especially in the facial area.

### BACKGROUND OF THE INVENTION

Cosmetic products or skin compositions, which improve the appearance of skin, are increasingly popular with consumers. For example, many consumers seek to minimize the “oily skin” look, resulting from sebum production in the sebaceous glands of the skin. Moreover, consumers also seek to alleviate or delay the signs of aged or photo-aged skin as well as dry and sagging skin. Further, consumers are concerned with the degree of pigmentation of their skin, whereby people with age spots or freckles often wish to eliminate or reduce the pronouncement of such spots. Others may wish to lighten their natural skin or reduce skin darkening caused by exposure to sunlight. To meet the needs of consumers, many attempts have been made to develop products that improve skin characteristics. The products developed thus far, however, tend to have low efficacy or undesirable side effects, such as, for example, toxicity or skin irritation. Additionally, known products are not always useful at improving a variety of negative skin characteristics, like those associated with oil, dryness and color.

There is an increasing interest to develop a skin benefit composition that, at the very least, decreases the impact of sebum secretion from sebocytes, especially in the facial area. This invention, therefore, is directed to a skin benefit composition. The skin benefit composition of the present invention surprisingly decreases the impact of sebum secretion from sebocytes and can moisturize and lighten skin. Moreover, the skin benefit composition of the present invention is not sticky or tacky, adheres to skin well, and does not create a draggy sensation when applied. The same comprises a mixture of fatty acids whereby at least about 40% of the total weight of fatty acids used in the composition are C₁₇ or less.

Additional Information

Efforts have been disclosed for making anti-sebum skin care cosmetic compositions. In European Patent EP 1181007 B1, anti-sebum skin care cosmetic compositions with branched esters are described.

Other efforts have been disclosed for alleviating oily skin. In U.S. Application Publication No. 2005/0079144 A1, methods for decreasing sebum production are described.

Still other efforts have been disclosed for treating skin. In International Application No. WO 94/27569, a skin treatment method that utilizes a composition and a pad is described.

None of the additional information above describes a skin benefit composition that, at the very least and surprisingly, decreases the impact of sebum secretion from sebocytes wherein the same comprises a mixture of fatty acids and where at least about 40% of the total weight of fatty acids used in the composition are C₁₇ or less.

### SUMMARY OF THE INVENTION

In a first aspect, the present invention is directed to a skin benefit composition comprising:

(a) a mixture of fatty acids, the mixture comprising at least about 40% fatty acids that are C₁₇ or less; and

(b) counterion

wherein from about 6 to about 20% on a mole basis of all fatty acid present within the mixture is neutralized.

In a second aspect, the present invention is directed to a method for treating skin with the skin benefit composition of the first aspect of this invention.

Skin benefit composition, as used herein, is meant to include cosmetic compositions suitable for use with humans and suitable to positively enhance a skin characteristic. Such a composition may generally be classified as leave-on or rinse-off and is meant to include hair care compositions like shampoos, conditioners or tonics; lipsticks; color cosmetics as well as a composition that reduces the impact of sebum, moisturizes and/or lightens.

The composition of the present invention can be in the form of a liquid, lotion, cream, gel, soap bar, toner or face mask. The preferred composition is one that, at the very least, delivers an anti-sebum benefit to the skin, where skin is meant to include the skin on the face, neck, chest, back, arms, hands, legs and scalp. All ranges identified herein are meant to implicitly include all ranges subsumed therein if, for example, reference to the same is not explicitly made.

### DETAILED DESCRIPTION OF THE PREFERRED EMBODIMENTS

The only limitation with respect to the types of fatty acids that may be used in this invention is that the fatty acids are suitable for use in a skin benefit composition. General examples of the types of fatty acids suitable for use in this invention include C₁₀ to C₃₀ fatty acids, all of which are generally classified as cosmetically acceptable carriers. Often, the fatty acids employed in this invention are selected from pelargonic, lauric, myristic, palmitic, stearic, isostearic, hydroxystearic, oleic, linoleic, ricinoleic, arachidic, behenic, erucic acid, as well as mixtures thereof or the like. Typically, the fatty acid employed in this invention is a mixture of fatty acids wherein at least about 40% of the fatty acid employed (based on total weight of fatty acid) is C₁₇ or less. In a preferred embodiment, at least about 95%, and preferably, about 100% of all fatty acid employed that is C₁₇ or less, is at least C₁₂-C₁₄ or greater. In yet another preferred embodiment, no more than about 90%, and preferably, no more than about 80% by weight of all fatty acid used in the mixture of fatty acids is C₁₇ or less. Yet, still in another preferred embodiment, from about 8.5 to about 60%, and preferably, from about 15 to about 35%, and most preferably, from about 25 to about 35% by weight of all fatty acid employed in the mixture of fatty acids is C₁₈-C₂₀.

In an especially preferred embodiment, however, the mixture of fatty acids has from about 25 to about 85% by weight fatty acid that is C₁₂-C₁₄, and from about 4.5 to about 45% by weight fatty acid that is C₁₅-C₁₇, and from about 8.5 to about 60% by weight fatty acid that is C₁₈-C₂₀, based on total weight of fatty acid used and including all ranges subsumed therein. In still another especially preferred embodiment, the mixture of fatty acids comprises lauric, myristic and palmitic acids or myristic, palmitic and stearic acids, and it is within the scope of the invention for the mixture to consist essentially of or consist of the same.

Typically, however, the total amount of fatty acid used in the skin benefit composition of the present invention is from about 2 to about 30%, and preferably from about 4 to about 20%, and most preferably, from about 8 to about 14% by weight, based on total weight of the skin benefit composition and including all ranges subsumed therein.

Turning to the counterion, the same is one which is suitable to neutralize the carboxylic acid group of any fatty acid used to make the mixture of fatty acids of this invention, and suitable for use in a skin benefit composition. Illustrative and non-limiting examples of the types of counterion that may be used in this invention include sodium hydroxide, potassium hydroxide, mono-, di, or tri-alkanolamines, trisodium ethylenediaminetetraacetic acid, mixtures thereof or the like. The preferred counterion used in this invention is an alkanolamine, and the most preferred is triethanolamine.

Typically, from about 6 to about 20%, and preferably, from about 7 to about 16%, and most preferably, from about 8 to about 12% on a mole basis of all fatty acid used within the mixture of fatty acids is neutralized by the counterion. Therefore, usually from about 0.25 to about 1.35% by weight (based on total weight of the skin benefit composition) counterion is used.

Not to be bound by theory, the mixture of fatty acids used in the skin benefit composition of this invention surprisingly decreases the impact of sebum secretion by, for example, absorbing the sebum on the user's skin. Therefore, the skin benefit composition of this invention can comprise, consist essentially of or consist of the mixture of fatty acids. Preferably, however, the skin benefit composition of the present invention comprises the mixture of fatty acids whereby the mixture of fatty acids also contributes to the composition by further acting as a commercially acceptable vehicle.

It should be known, however, that if desired, commercially acceptable and conventional vehicles (in addition to mixture of fatty acids of the present invention) may be used, acting as diluants, dispersants or carriers for optional but often preferred additives. Therefore, the vehicle of the skin benefit composition described herein may be aqueous-based, anhydrous or an emulsion whereby a water-in-oil or oil-in-water emulsion is generally preferred. If the use of water is desired, water typically makes up the balance of the skin benefit composition, and preferably, makes up from about 5 to about 98%, and most preferably, from about 65 to about 90% by weight of the skin benefit composition, including all ranges subsumed therein.

In addition to water, organic solvents may be optionally included to act or assist as carriers within the compositions of the present invention. Illustrative and non-limiting examples of the types of organic solvents suitable for use in the present invention include alkanols like methyl, ethyl and isopropyl alcohol, mixtures thereof or the like.

Other optional additives suitable for use include ester oils like isopropyl myristate, cetyl myristate, 2-octyldodecyl myristate, avocado oil, almond oil, olive oil, neopentylglycol dicaprate, mixtures thereof or the like. Typically such ester oils assist in emulsifying the skin benefit composition of this invention, and an effective amount is often used to yield a stable, and most preferably, water-in-oil emulsion.

Emollients may also be used, if desired, in the skin benefit composition of the present invention (optionally, from about 0.1 to about 50% by weight of the total weight of the skin benefit composition). Alcohols like 1-hexadecanol (i.e., cetyl alcohol) and phenoxyethanol are often desired as are the emollients generally classified as silicone oils and synthetic esters. Silicone oils suitable for use include cyclic or linear polydimethylsiloxanes containing from 3 to 9, preferably from 4 to 5, silicon atoms. Linear volatile silicone materials generally have viscosities less than about 5 centistokes at 25° C. while cyclic materials typically have viscosities of less than about 10 centistokes. Nonvolatile silicone oils useful as an emollient material in the inventive skin benefit composition described herein include polyalkyl siloxanes, polyalkylaryl siloxanes and polyether siloxane copolymers. The essentially non-volatile polyalkyl siloxanes useful herein include, for example, polydimethylsiloxanes with viscosities of from about 5 to about 25 million centistokes at 25° C. Among the preferred non-volatile emollients useful in the present compositions are the polydimethylsiloxanes having viscosities from about 10 to about 400 centistokes at 25° C.

The ester emollients that may optionally be used are:
(1) alkenyl or alkyl esters of fatty acids having 10 to 20 carbon atoms. Examples thereof include isoarachidyl neopentanoate, isononyl isonanonoate, oleyl myristate, oleyl stearate, and oleyl oleate.
(2) ether-esters such as fatty acid esters of ehtoxylated fatty alcohols.
(3) polyhydric alcohol esters. Ethylene glycol mono and di-fatty acid esters, diethylene glycol mono-and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty esters, ethoxylated glyceryl mono-stearate, 1,3-butylene glycol monostearate, 1,3-butylene glycol distearate, polyoxyethylene polyol fatty acid ester, sorbitan fatty acid esters, and polyoxyethylene sorbitan fatty acid esters are satisfactory polyhydric alcohol esters.
(4) wax esters such as beeswax, spermaceti, stearyl stearate and arachidyl behenate.
(5) sterols esters, of which cholesterol fatty acid esters are examples.

Emollients are optional, but can make up from about 0.1 to bout 50% by weight of the skin benefit composition, including all ranges subsumed therein.

Humectants of the polyhydric alcohol type may also be employed in the skin benefit compositions of this invention. The humectant often aids in increasing the effectiveness of the emollient, reduces scaling, stimulates removal of built-up scale and improves skin feel. Typical polyhydric alcohols include glycerol, polyalkylene glycols and more preferably alkylene polyols and their derivatives, including propylene glycol, dipropylene glycol, polypropylene glycol, polyethylene glycol and derivatives thereof, sorbitol, hydroxypropyl sorbitol, hexylene glycol, 1,3-butylene glycol, 1,2,6-hexanetriol, ethoxylated glycerol, propoxylated glycerol and mixtures thereof. For best results the humectant is preferably propylene glycol or sodium hyaluronate. The amount of humectant may range anywhere from 0.2 to 15%, and preferably, from about 0.5 to about 3.0% by weight of the skin benefit composition, including all ranges subsumed therein.

Thickeners may also be utilized as part of the cosmetically acceptable carrier in the skin benefic compositions of the present invention. Typical thickeners include cross linked acrylates (e.g. Carbopol 982), hydrophobically-modified acrylates (e.g. Carbopol 1382), cellulosic derivatives and natural gums. Among useful cellulosic derivatives are sodium carboxymethylcellulose, hydroxypropyl methylcellulose, hydroxypropyl cellulose, hydroxyethyl cellulose, ethyl cellulose and hydroxymethyl cellulose. Natural gums suitable for the present invention include guar, xanthan, sclerotium, carrageenan, pectin and combinations of these gums. Amounts of the thickener may range from 0.0001 to 5%, usually from 0.001 to 1%, optimally from 0.01 to 0.5% by weight.

Collectively, the water, solvents, silicones, esters, fatty acids, humectants and/or thickeners will constitute the cosmetically acceptable carrier in amounts from 1 to 99.9%, preferably from 80 to 99% by weight.

Various types of optional additional active ingredients may be used in the skin benefit compositions of the present invention. Actives are defined as skin benefit agents other than emollients and other than ingredients that merely improve the physical characteristics of the composition. Although not limited to this category, general examples include additional anti-sebum ingredients such as talcs and silicas, as well as alpha-hydroxy acids, beta-hydroxy acids, poly-hydroxy acids, benzyl peroxide, zinc salts, sunscreens.

Beta-hydroxy acids include salicylic acid, for example. Zinc pyrithione is an example of the zinc salts useful in the skin benefit composition of the present invention.

Sunscreens include those materials commonly employed to block ultraviolet light. Illustrative compounds are the derivatives of PABA, cinnamate and salicylate. For example, avobenzophenone (Parsol 1789®) octyl methoxycinnamate and 2-hydroxy-4-methoxyl benzophenone (also known as oxybenzone) can be used. Octyl methoxycinnamate and 2-hydroxy-4-methoxy benzophenone are commercially available under the trademarks, Parsol MCX and Benzophenone-e, respectively. The exact amount of sunscreen employed in the compositions can vary depending upon the degree of protection desired from the sun's UV radiation. Additives that reflect or scatter the suns rays may also be employed. These additives include oxides like zinc oxide and titanium dioxide.

Many cosmetic compositions, especially those containing water, should be protected against the growth of potentially harmful microorganisms. Anti-microbial compounds, such as triclosan, and preservatives are, therefore, typically necessary. Suitable preservatives include alkyl esters of p-hydroxybenzoic acid, hydantoin derivatives, propionate salts, and a variety of quaternary ammonium compounds. Particularly preferred preservatives of this invention are methyl paraben, propyl paraben, phenoxyethanol and benzyl alcohol. Preservatives will usually be employed in amounts ranging from about 0.1% to 2% by weight of the composition.

Still other optional ingredients that may be used with the skin benefit composition of this invention include vitamins, like niacinamide, recorcinols and retinoids, including retinoic acid, retinal, retinal and retinyl esters.

The skin benefit compositions of the present invention are intended for use primarily as a product for topical application to human skin, especially as an agent for controlling the impact of sebum secretion on the skin, moisturizing skin and physically making the skin look lighter. Often, the skin benefit composition of the present invention has a melting point from about 30° C. to about 45° C., including all ranges subsumed therein. When the lower melting point composition is desired, the fatty acids used are typically C₁₂-C₁₈ whereby when the higher melting point composition is desired C₁₄-C₁₈ fatty acids are typically used. When moisturizing is the primary focus, C₁₂-C₁₈ fatty acids are generally preferred. When oil control is the primary focus, C₁₄-C₁₈ fatty acids are generally preferred.

When making the skin benefit composition of the present invention, the desired ingredients are mixed in no particular order and usually at temperatures from about 70 to about 80° C. and under atmospheric pressure.

The packaging for the skin benefit composition of this invention can be a bottle, tube, roll-ball applicator, propellant driven aerosol device, squeeze container or lidded jar.

The examples below are provided to illustrate the invention and are not intended to limit the scope of the claims.

### EXAMPLE 1

Skin benefit compositions comprising a mixture of fatty acids in parts by weight and counterion (as shown in Table 1) were prepared by mixing counterion, water and fatty acid. Homogeneous mixtures were produced by mixing the ingredients for about five (5) minutes at about 75° C.

TABLE 1

| Ingredient | A | B | C | D | E |
|---|---|---|---|---|---|
| Lauric acid | 7.75 | 5.17 | 7.75 | 7.75 | — |
| Myristic acid | 4.76 | 3.17 | 4.76 | 4.76 | — |
| Palmitic acid | 2.49 | 6.66 | 2.49 | 2.49 | — |
| Stearic acid | — | — | — | — | 18 |
| Triethanolamine | 10* | 10* | 15* | 20* | 10* |
| (*mole percent | | | | | |
| neutralization of | | | | | |
| fatty acid) | | | | | |
| Water | Balance | Balance | Balance | Balance | Balance |

Uniform films of about 75 μm thickness were drawn on plastic from Aclar Embedding Film (film dimensions circa 10 cm²) for each of the above-defined skin benefit compositions. A sheen applicator was used. The resulting films were allowed to dry overnight and about 1 μm of sebum was injected on the film using a syringe injector. Spreading of the sebum was observed under a microscope, and images of the sebum spreading were taken at 0, 3, 5, 10, 15, 20, 25, 30, 45, 60 and 100 minute intervals. To calculate the area occupied by sebum, the images were analyzed using image processing software. The greater area of sebum spreading or dissipation indicates that the skin benefit compositions of the present invention are surprisingly superior for reducing the impact of sebum especially when compared to a control with stearic acid. A summary of the results is shown in Table 2.

TABLE 2

| Formula | Time (min.) | Area (cm²) |
|---|---|---|
| A | 20 | 0.20 |
| A | 60 | 0.28 |
| A | 100 | 0.38 |
| B | 20 | 0.05 |
| B | 60 | 0.06 |
| B | 80 | 0.06 |
| C | 20 | 0.14 |
| C | 60 | 0.13 |
| C | 100 | 0.12 |
| D | 20 | 0.13 |
| D | 60 | 0.12 |
| D | 100 | 0.11 |
| E | 20 | 0.02 |
| E | 60 | 0.02 |
| E | 80 | 0.02 |

TABLE 3

| Ingredient | F | G |
|---|---|---|
| Lauric acid | 7.75 | — |
| Myristic acid | 4.76 | — |
| Palmitic acid | 2.49 | — |
| Stearic acid | — | 18 |
| Counterion/mole percent | KOH/10% | KOH/10% |
| fatty acid neutralization | | |
| Water | Balance | Balance |

Additional compositions represented by formulae F and G in Table 3 were made into films (about 75 μm thick, 10 cm²). The films were left to dry at room temperature. A digital camera was used to analyze resulting images. The images demonstrated that the skin benefit composition made consistent with this invention (formula F) was easier to handle, more consistent and less draggy than conventional formulas made with stearic acid (formula G).

### EXAMPLE 2

Skin benefit compositions consistent with this invention were made by mixing the following ingredients for about five (5) minutes at about 75° C.:

| Ingredient | % by Weight |
|---|---|
| Myristic acid | 5.2 |
| Stearic acid | 1.7 |
| Palmitic acid | 3.2 |
| Triethanolamine | 0.6 |
| Alcohol | 0.7 |
| Emulsifier | 0.8 |
| Sunscreen | 1.3 |
| Silicone | 0.5 |
| TiO₂ | 0.2 |
| EDTA | 0.04 |
| Humectant | 1.0 |
| Preservative | 0.2 |
| Vitamin | 1.3 |
| Water | Balance |

Control compositions were made with 18% by weight stearic acid in lieu of the mixture of fatty acids described above and with 8% by weight less water.

Twenty panelists were asked to compare the skin benefit compositions of this invention to the control composition with stearic acid by applying both to their faces (bilaterally). All panelists concluded that the compositions made according to this invention were better at moisturizing and reducing an oily sensation when compared to the control.

Moreover, all panelists concluded that the skin benefit compositions of this invention lightened their skin (from a physical standpoint) to a degree at least consistent with that of the control, and that the compositions of this invention were less sticky and draggy and adhered to the skin better (when applied at room temperature) when compared to the control.

### EXAMPLE 3

Skin benefit compositions consistent with this invention were made by mixing the following ingredients for about five (5) minutes at about 75° C.:

| Ingredient | % by Weight |
|---|---|
| Lauric acid | 5.2 |
| Palmitic acid | 1.7 |
| Myristic acid | 3.2 |
| Triethanolamine | 0.6 |
| Alcohol | 0.7 |
| Emulsifier | 0.8 |
| Sunscreen | 1.3 |
| Silicone | 0.5 |
| TiO₂ | 0.2 |
| EDTA | 0.04 |
| Humectant | 1.00 |
| Preservative | 0.2 |
| Vitamin | 1.3 |
| Water | Balance |

Three distinct control compositions were made with 18% by weight stearic acid, 18% by weight sunflower seed oil and 18% by weight petrolatum in lieu of the mixture of fatty acids described above and with 8% by weight less water.

Twenty panelists were asked to compare the skin benefit compositions of this invention to the control compositions with stearic acid and cosmetic oil (in a manner similar to the one described in Example 2). All panelists concluded that the compositions made according to this invention were better at moisturizing their skin than the controls. The panelists also concluded that the compositions of this invention were less sticky during application and less draggy when compared to the controls.

Moreover, the panelists concluded that the compositions of this invention adhered better to the skin when compared to the controls.

What is claimed is:

1. A skin benefit composition comprising:
(a) a mixture of fatty acids, the mixture comprising at least about 40% by weight fatty acids that are C₁₇ or less; and
(b) counterion
wherein from about 6 to about 20% on a mole basis of all fatty acid present within the mixture is neutralized.

2. The skin benefit composition according to claim 1 wherein at least about 95% of all fatty acid that is C₁₇ or less is C₁₂ or greater.

3. The skin benefit composition according to claim 1 wherein at least about 8.5 to about 60% by weight of fatty acid in the mixture of fatty acids is C₁₈-C₂₀.

4. The skin benefit composition according to claim 1 wherein at least about 95% by weight of all fatty acid that is C₁₇ or less is C₁₄ or greater.

5. The skin benefit composition according to claim 1 wherein from about 25 to about 35% by weight of all fatty acid is C₁₈-C₂₀.

6. The skin benefit composition according to claim 1 wherein from about 25 to about 85% by weight fatty acid used is C₁₂-C₁₄, from about 4.5 to about 45% by weight fatty acid used is C₁₅-C₁₇ and from about 8.5 to about 60% by weight fatty acid used is C₁₈-C₂₀.

7. The skin benefit composition according to claim 1 wherein the counter ion is NaOH, KOH, a mono-, di- or tri-alkanolamine, ethylenediaminetetraacetic acid, or a mixture thereof.

8. The skin benefit composition according to claim 7 wherein the counter ion is a trialkanolamine and the trialkanolamine is triethanolamine.

9. The skin benefit composition according to claim 1 wherein from about 7 to about 16% on a mole basis of all fatty acid used is neutralized.

10. The skin benefit composition according to claim 1 wherein from about 8 to about 12% on a mole basis of all fatty acid used is neutralized.

11. The skin benefit composition according to claim 1 wherein the skin benefit composition is an emulsion.

12. The skin benefit composition according to claim 1 wherein the skin benefit composition further comprises an emollient, water, humectant, thickener, organic solvent, ester oils, sunscreen or a mixture thereof.

13. The skin benefit composition according to claim 1 wherein the skin benefit compositon further comprises talc, silica, hydroxy acid, benzylperoxide, zinc salts, zinc oxide, titanium dioxide preservatives, vitamin, recorcinol, retinoid or a mixture thereof.

14. The skin benefit composition according to claim 1 wherein the skin benefit composition has a melting point from about 30° C. to about 45° C.

15. A method for moisturizing skin, lightening skin and/or reducing the effects of sebum on the skin, the method comprising the steps of:
(a) obtaining a skin benefit composition comprising:
i. a mixture of fatty acids, the mixture comprising at least about 40% by weight fatty acids that are C₁₇ or less; and
ii. counterion; and
(b) contacting the skin with the skin benefit composition
wherein from about 6 to about 20% on a mole basis of all fatty acid present within the mixture is neutralized.

16. The method according to claim 15 wherein skin moisturizing is achieved and the mixture of fatty acids comprise C₁₂-C₁₈ fatty acids.

17. The method according to claim 15 wherein the effects of sebum on the skin are reduced and the fatty acids comprise C₁₄-C₁₈ fatty acids.
